(19) United States
(12) Patent Application Publication
(10) Pub. No.: US 2022/0061993 A1
(43) Pub. Date: Mar. 3, 2022

(54) THREE-DIMENSIONAL IMPLANT USED IN CRANIOPLASTY AND METHOD FOR MANUFACTURING SAME

(71) Applicant: INDUSTRY-ACADEMIC COOPERATION FOUNDATION, YONSEI UNIVERSITY, Seoul (KR)

(72) Inventor: Kyuwon SHIM, Seoul (KR)

(73) Assignee: INDUSTRY-ACADEMIC COOPERATION FOUNDATION, YONSEI UNIVERSITY, Seoul (KR)

(21) Appl. No.: 17/422,439

(22) PCT Filed: Jan. 28, 2020
(86) PCT No.: PCT/KR2020/001249

§ 371 Date: Jul. 12, 2021

(30) Foreign Application Priority Data
Jan. 28, 2019 (KR) 10-2019-0010388

(51) Int. Cl. A61F 2/28; A61F 2/30

(52) U.S. Cl. CPC A61F 2/2875; A61F 2/30749; A61F 2310/00023; A61F 2002/2882; A61F 2002/30985; A61F 2002/30948

(57) ABSTRACT

In a three-dimensional implant and a method for manufacturing the three-dimensional implant, the three-dimensional implant is manufactured based on an image on a defect area of a skull. The image is generated based on a computed tomography on the skull of a patient. The three-dimensional implant includes a body portion, an extended portion and a fixing portion. The body portion has a shape substantially same as the defect area of the skull. The extended portion is disposed at a front of the body portion, is fixed to a zygomaticofrontal suture of the patient, and has a thickness larger than that of the body portion. The fixing portion is protruded along an outline of the body portion and is fixed with the skull.

### BACKGROUND

### 1. Field of Disclosure

The present disclosure of invention relates to a three-dimensional implant and a method for manufacturing the same, and more specifically the present disclosure of invention relates to a three-dimensional implant and a method for manufacturing the same, capable of replacing skull used in cranioplasty for a defect area.

### 2. Description of Related Technology

In cases that the skull is partially defected such as skull amputation, skull depression and so on due to an external wound or congenital anomalies, or in cases that cranioplasty for decreasing a pressure in a cranial cavity is performed to cause the partial defect of the skull, the cranioplasty should be performed for the defect area.

Conventionally, an implant has been used for the cranioplasty for the defect area, and the conventional implant is illustrated in FIG. 1.

However, referring to FIG. 1, in cases that the conventional implant is used for the cranioplasty, a soft tissue is damaged to cause temporalis muscle hollowing 20, that is a depression problem, when a muscle is separated from periosteum. Here, the temporalis muscle hollowing 20 decreases patient satisfaction and causes a bad prognosis in a brain function or a brain exterior. Thus, the temporalis muscle hollowing 20 should be solved.

However, until now, in manufacturing the implant used in the cranioplasty, to manufacturing the implant considering the size or the shape of the defect area is the most important. In addition, the implant is merely manufactured via considering or copying a structure of a shape of a non-defect area which is an opposite to the defect area.

Related prior art is Japanese laid-open patent No. 2017-74293.

### SUMMARY

The present invention is developed to solve the above-mentioned problems of the related arts. The present invention provides a three-dimensional implant capable of preventing temporalis muscle hollowing in cranioplasty for a defect area of a skull using an implant.

In addition, the present invention also provides a method for manufacturing the three-dimensional implant.

According to an example embodiment, the three-dimensional implant is manufactured based on an image on a defect area of a skull. The image is generated based on a computed tomography on the skull of a patient. The three-dimensional implant includes a body portion, an extended portion and a fixing portion. The body portion has a shape substantially same as the defect area of the skull. The extended portion is disposed at a front of the body portion, is fixed to a zygomaticofrontal suture of the patient, and has a thickness larger than that of the body portion. The fixing portion is protruded along an outline of the body portion and is fixed with the skull.

In an example, a surface of the extended portion may be protruded, so that a position of the surface of the extended portion may be maintained to be the same as a protruded position of zygomatic bone of the patient.

In an example, the extended portion may be inserted between a superficial temporalis muscle of the patient and a deep temporalis muscle of the patient.

In an example, the body portion, the extended portion and the fixing portion may be integrally formed with each other.

In an example, each of the body portion, the extended portion and the fixing portion may include polymethylmethacrylate (PMMA) or titanium.

According to another example embodiment, in the method for manufacturing a three-dimensional implant, a computed tomography information on a skull of a patient is obtained. A skull image is generated based on the computed tomography information. An image on a body portion which is a defect area of the skull is generated, based on a normal portion of the skull image. An additional image on an extended portion which is fixed to a zygomaticofrontal suture of the patient is generated, in addition to the generated image on the body portion. An implant having the body portion and the extended portion is manufactured via a three-dimensional printer.

In an example, in generating the additional image, the additional image may be generated such that a thickness of the extended portion is larger than that of the body portion.

In an example, in manufacturing the implant, the body portion and the extended portion may be integrally manufactured using a material of polymethylmethacrylate (PMMA) or titanium.

According to the present example embodiments, in the three-dimensional implant used in the cranioplasty, the extended portion fixed to the zygomaticofrontal suture of the patient is added to have a thickness larger than that of the body portion. Thus, temporalis muscle hollowing, that is a depression of temporalis muscle, generated in cranioplasty which is performed by the conventional implant, may be prevented. Thus, a brain function or a brain exterior problem generated after the implant insertion may be minimized.

Here, a position of the surface of the extended portion is maintained to be the same as a protruded position of zygomatic bone of the patient, so that appearance satisfaction of the patient may be increased in addition to the preventing of the depression.

In addition, the extended portion is integrally formed or manufactured with the body portion, using the three-dimensional printer, so that the manufacturing may be more simplified, the stiffness of the implant may be increased, and the durability and the efficiency may be increased.

### BRIEF DESCRIPTION OF THE DRAWINGS

FIG. 1 is a perspective view illustrating a conventional implant used in cranioplasty;

FIG. 2 is a perspective view illustrating a three-dimensional implant used in cranioplasty according to an example embodiment of the present invention;

FIG. 3 is a perspective view illustrating an extended portion in the implant of FIG. 2;

FIG. 4 is a flow chart showing a method for manufacturing the implant of FIG. 2;

FIG. 5A, FIG. 5B, FIG. 5C, FIG. 5D, FIG. 5E and FIG. 5F are images showing steps of cranioplasty using the implant of FIG. 2; and

FIG. 6 shows a result of cranioplasty using the implant of FIG. 2 compared to cranioplasty using the conventional implant.

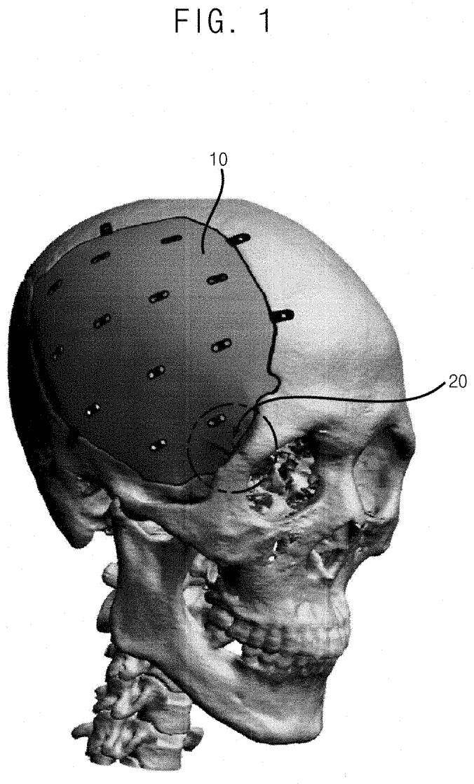

* Reference numerals

| | |
|---|---|
| 100: three-dimensional implant | 110: body portion |
| 120: extended portion | 130: fixing portion |

### DETAILED DESCRIPTION

The invention is described more fully hereinafter with Reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention. Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms (including technical and scientific terms) used herein have the same meaning as commonly understood by one of ordinary skill in the art to which this invention belongs.

It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, example embodiments of the present invention are explained in detail referring to the figures.

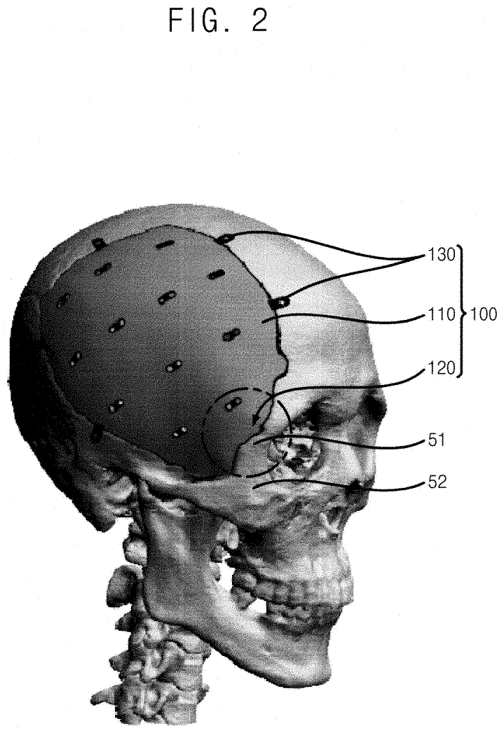

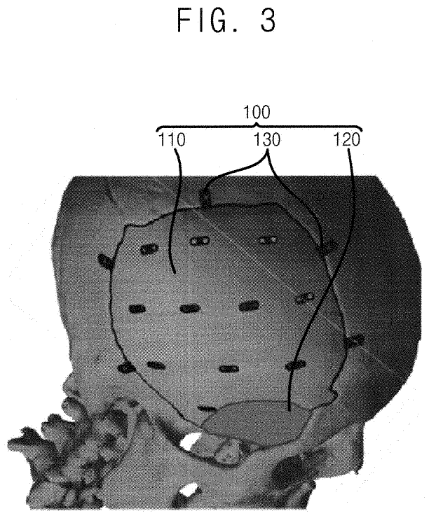

FIG. 2 is a perspective view illustrating a three-dimensional implant used in cranioplasty according to an example embodiment of the present invention. FIG. 3 is a perspective view illustrating an extended portion in the implant of FIG. 2.

Referring to FIG. 2 and FIG. 3, the three-dimensional implant 100 (hereinafter, the implant) is an implant used in cranioplasty, and is manufactured to match a defect area of a skull.

Here, the implant 100 includes a body portion 110, an extended portion 120 and a fixing portion 130.

The defect area of the skull may variously occur according to the patient, but the defect area may be normally generated in a portion of one of side portions of the skull. Thus, the implant 100 may replace the defect area of the skull generated in the portion of one of the side portions of the skull.

The implant 100 according to the present invention is applied to the case that the defect area is generated to be extended to a zygomatic bone. As illustrated in FIG. 2, the implant 100 may be applied to the case that the defect area is generated to zygomaticofrontal suture which is positioned at an upper side of the zygomatic bone 52.

Accordingly, the body portion 110 is formed to have a boundary surface, that is an outer surface, substantially same as a boundary surface along the defect area of the skull. In addition, a thickness of the body portion 110 is substantially same as that of the skull.

Thus, the body portion 110 is entirely formed to have an outer shape substantially same as that of the skull which is not defected. Here, as explained below, an image is hard to be generated to be the same as the shape of the skull before the defect, so that the shape of the defect area may be generated referring to the shape of the skull which is not defected and is an opposite side of the defect area. Thus, the body portion 110 is formed to have the shape substantially same as or very similar with the shape of the skull which is not defected and is the opposite side of the defect area.

The extended portion 120 is disposed at a front side of the body portion 110, and the extended portion 120 is extended from the body portion 110 as a portion of the body portion 110. Thus, the extended portion 120 is integrally formed with the body portion 110.

The extended portion 120 is a portion included in the body portion 110, but a thickness of the extended portion 120 is different from that of the body portion 110.

As explained above, the body portion 110 is formed to have the thickness substantially same as that of the skull of the patient, but the extended portion 120 is formed to have the thickness larger than that of the body portion 110. That means that the extended portion 120 is reinforced.

Here, the extended portion 120 is positioned at the front side of the body portion 110, and is a portion fixed to the zygomaticofrontal suture of the patient.

The extended portion 120 has an outer line combined with the defect area corresponding to zygomaticofrontal suture, in the defect area, and the extended portion 120 has a predetermined area from the outer line.

For example, the area of the extended portion 120 may be less than 10% of an entire area of the body portion 110.

Normally, the zygomatic bone 52 of the patient is protruded a little from the skull, and the extended portion 120 in the present example embodiment has a protruded surface to be positioned substantially same as the position of the protruded portion of the zygomatic bone 52 of the patient.

The extended portion 120 is reinforced to have a predetermined thickness, to that the surface of the extended portion 120 has the position substantially same as the protruded portion of the zygomatic bone 52 of the patient. Thus, the thickness of the extended portion 120 is larger than that of the body portion 110.

The fixing portion 130 is formed as a plural according to the outer line of the body portion 110. The fixing portion 130 fixes the body portion 110 with the boundary surface of the defect area of the skull, so as to fix the implant 100 to the skull of the patient.

The body portion 110, the extended portion 120 and the fixing portion 130 are integrally formed or manufactured with each other, and may include polymethylmethacrylate (PMMA) or titanium.

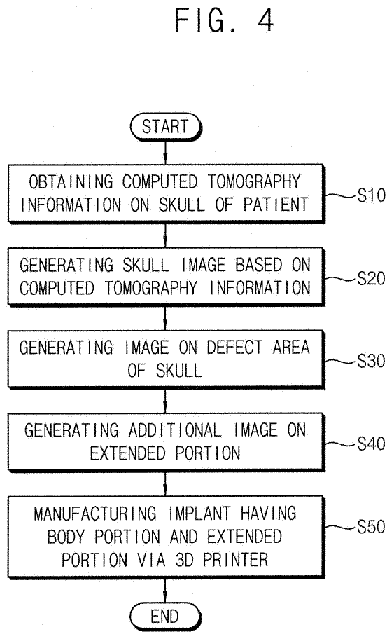

FIG. 4 is a flow chart showing a method for manufacturing the implant of FIG. 2.

Referring to FIG. 4, in the method for manufacturing the implant 100, a computed tomography (CT) is performed on the skull of the patient, and the computed tomography information is obtained (step S10).

Using the computed tomography information, front information, plane information and side information may be obtained, and thus the shape or the structure of the skull or the defect area of the skull may be obtained.

Then, based on the computed tomography information, an image on the skull of the patient is generated (step S20).

Here, to generate the computed tomography information to the image, the computed tomography information should be transformed to an image design program like a CAD, and then the image on the skull of the patient may be generated using the image design program.

Accordingly, the generated image on the skull may be as illustrated in FIG. 2 except for the implant 100, and the defect area may be illustrated as the image with the skull. Here, the generated image is generated based on the computed tomography information, and thus an actual skull image of the patient including the defect area is generated.

When the image is generated to include the defect area of the skull of the patient, the body portion 110 is sufficient to have the shape and the structure substantially same as those of the defect area. Thus, as illustrated in FIG. 2, the image on the body portion 110 may be generated at the same time.

However, in the present example embodiment, the extended portion 120 is formed at the front side of the body 110, and thus an additional image should be generated.

From the generated image on the defect area, an additional image on the extended portion 120 which is fixed to zygomaticofrontal suture is generated (step S40).

Here, in generating the additional image, the computed tomography information is transformed to the image design program like the CAD, and then the additional image is generated.

As explained above, the extended portion 120 has the outer line combined with the defect area corresponding to zygomaticofrontal suture of the patient, and the extended portion 120 corresponds to an area having the predetermined area from the outer line. Thus, an area of the extended portion 120 is generated as explained above, in generating the additional image.

In addition, the extended portion 120 has the protruded surface to maintain the position of the protruded surface of the extended portion 120 to be the same as the position of the protruded portion of the zygomatic bone 52 of the patient. Thus, by extracting the information on the zygomatic bone 52 from the computed tomography information, the image is generated to form the surface of the extended portion 120 to be protruded substantially same as that of the zygomatic bone 52 of the patient.

Accordingly, the additional image is generated to include the information on the area and the thickness of the extended portion 120.

Then, the implant 100 is manufactured based on the image on the body portion and the additional image on the extended portion, via a three-dimensional printer (step S50).

Here, the implant 100 may include polymethylmethacrylate (PMMA) or titanium, as explained above.

Accordingly, in manufacturing the implant 100 using the 3D printer, a laser may be irradiated to the material including polymethylmethacrylate (PMMA) or titanium, to form the implant.

In manufacturing the implant 100 using the 3D printer with the irradiation of the laser, an additive manufacturing may be used. In the additive manufacturing, the material is melted and stacked to form the entire shape of the implant 100.

For example, the implant 100 may include titanium, and here, the titanium is melted by a laser irradiating a heat over about 1,500˜2,000° C., and the titanium is stacked layer by layer, to form the implant 100 having the body portion 110 and the extended portion 120, using the 3D printer having a laser irradiation unit.

Accordingly, when the implant 100 includes a metal like the titanium, immune rejection may be prevented. In addition, the material may be sterilized due to the heat, and the sterilization may be additionally performed right before the surgery, to minimize the infection from the implant.

In addition, in manufacturing the implant with the metal material, the implant 100 is formed to have a porous structure or a honeycomb structure. Here, the implant is manufactured by the stack process, the porous structure or the honey comb structure may be formed more easily.

The implant 100 may be manufactured by forming the porous structure in the surface of the implant 100, or by forming the honeycomb structure through the implant 100.

Accordingly, since the implant 100 includes the porous or honeycomb structure, the implant 100 may be engrafted with a living tissue such as a tissue or a bone more easily. Thus, various kinds of living tissues may be engrafted with the implant 100 and thus the engrafted living tissues may be grown better.

In addition, since the implant 100 has the porous or honeycomb structure which is an opening portion, the implant 100 may be manufactured more lightly compared to the conventional implant without the porous or honeycomb structure. In addition, the material used in the manufacturing the implant 100 may be decreased too.

The implant 100 is manufactured to have a plurality of fixing portions 130 along the outer line of the body portion 110. Thus, the implant 100 may be easily and tightly fixed to the skull of the patient.

After the manufacturing of the implant 100 as illustrated in FIG. 2, the cranioplasty using the implant 100 may be performed as follows.

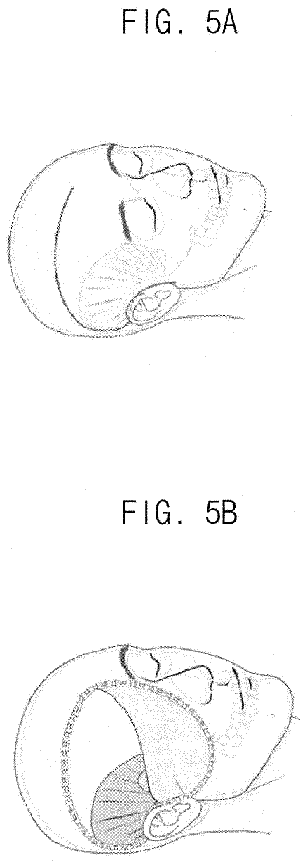

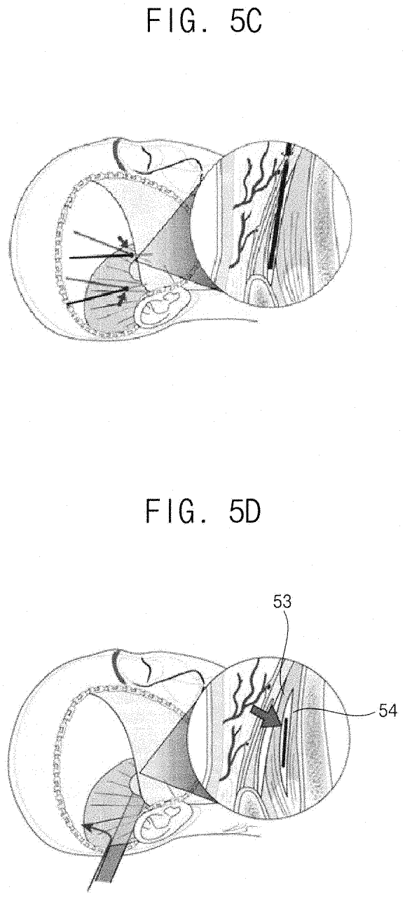

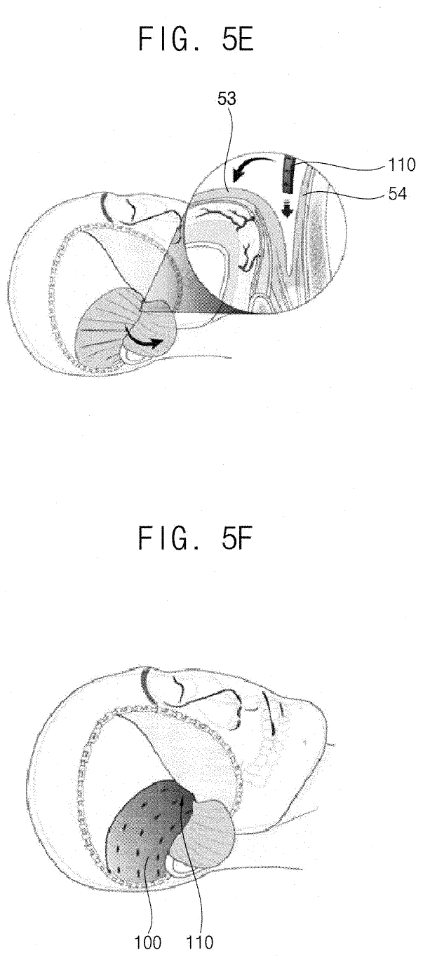

FIG. 5A, FIG. 5B, FIG. 5C, FIG. 5D, FIG. 5E and FIG. 5F are images showing steps of cranioplasty using the implant of FIG. 2.

Referring to FIG. 5A, in the cranioplasty using the implant 100, incision is performed along the defect area of the patient, and as illustrated in FIG. 5B, the incised defect area is incised to expose temporalis fat pad and scalp flap is fixed to be positioned at a front side of the patient.

Then, referring to FIG. 5C, dissection is performed on an interfacial at a front side and a rear side of a fat pad, and as illustrated in FIG. 5D, from a root of zygomatic arch, zygomaticofrontal suture superficial temporalis muscle 53 and deep temporalis muscle 54 are incised.

Then, as illustrated in FIG. 5E, the superficial temporalis muscle 53 and the deep temporalis muscle 54 are separated, and the superficial temporalis muscle 53 is positioned to an ear side of the patient.

Then, referring to FIG. 5E and FIG. 5F, the implant 100 is positioned at the incised position, and here, the extended portion 120 is inserted between the superficial temporalis muscle 53 and the deep temporalis muscle 54, so that the extended portion 120 is fixed to zygomaticofrontal suture.

Accordingly, the implant 100 having the extended portion 120 is positioned at the defect area of the patient, and the implant 100 is fixed with the defect area of the patient via the fixing portion 130. Then, the cranioplasty for the defect area is finished.

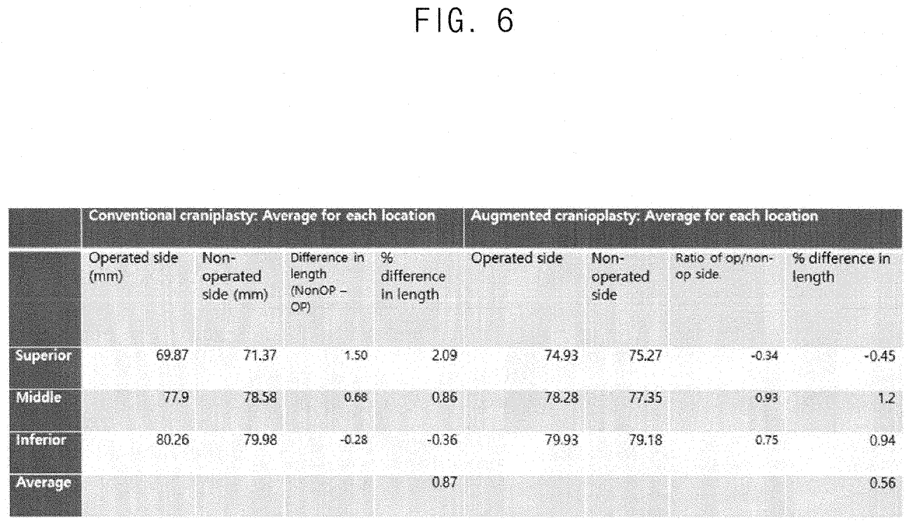

FIG. 6 shows a result of cranioplasty using the implant of FIG. 2 compared to cranioplasty using the conventional implant.

As illustrated in FIG. 6, difference in length at each position after conventional cranioplasty using the conventional implant is larger than that after augmented cranioplasty using the implant 100 of the present example embodiment.

Accordingly, as in the present example embodiment, the implant 100 having the extended portion 120 is used in cranioplasty for the defect area, to increase an appearance distinction and to prevent the depression of the brain.

According to the present example embodiment, in the three-dimensional implant used in the cranioplasty, the extended portion fixed to the zygomaticofrontal suture of the patient is added to have a thickness larger than that of the body portion. Thus, temporalis muscle hollowing, that is a depression of temporalis muscle, generated in cranioplasty which is performed by the conventional implant, may be prevented. Thus, a brain function or a brain exterior problem generated after the implant insertion may be minimized.

Here, a position of the surface of the extended portion is maintained to be the same as a protruded position of zygomatic bone of the patient, so that appearance satisfaction of the patient may be increased in addition to the preventing of the depression.

In addition, the extended portion is integrally formed or manufactured with the body portion, using the three-dimensional printer, so that the manufacturing may be more simplified, the stiffness of the implant may be increased, and the durability and the efficiency may be increased.

Although the exemplary embodiments of the present invention have been described, it is understood that the present invention should not be limited to these exemplary embodiments but various changes and modifications can be made by one ordinary skilled in the art within the spirit and scope of the present invention as hereinafter claimed.

What is claimed is:

1. A three-dimensional implant manufactured based on an image on a defect area of a skull, the image being generated based on a computed tomography on the skull of a patient, the three-dimensional implant comprises:
a body portion having a shape substantially same as the defect area of the skull;
an extended portion disposed at a front of the body portion, fixed to a zygomaticofrontal suture of the patient, and having a thickness larger than that of the body portion; and
a fixing portion protruded along an outline of the body portion and fixed with the skull.

2. The three-dimensional implant of claim 1, wherein a surface of the extended portion is protruded, so that a position of the surface of the extended portion is maintained to be the same as a protruded position of zygomatic bone of the patient.

3. The three-dimensional implant of claim 2, wherein the extended portion is inserted between a superficial temporalis muscle of the patient and a deep temporalis muscle of the patient.

4. The three-dimensional implant of claim 1, wherein the body portion, the extended portion and the fixing portion are integrally formed with each other.

5. The three-dimensional implant of claim 4, wherein each of the body portion, the extended portion and the fixing portion comprises polymethylmethacrylate (PMMA) or titanium.

6. A method for manufacturing a three-dimensional implant, the method comprising:
obtaining a computed tomography information on a skull of a patient;
generating a skull image based on the computed tomography information;
generating an image on a body portion which is a defect area of the skull, based on a normal portion of the skull image;
generating an additional image on an extended portion which is fixed to a zygomaticofrontal suture of the patient, in addition to the generated image on the body portion; and
manufacturing an implant having the body portion and the extended portion, via a three-dimensional printer.

7. The method of claim 6, wherein in generating the additional image,
the additional image is generated such that a thickness of the extended portion is larger than that of the body portion.

8. The method of claim 8, wherein in manufacturing the implant,
the body portion and the extended portion are integrally manufactured using a material of polymethylmethacrylate (PMMA) or titanium.
